(19) United States
(12) Patent Application Publication
(10) Pub. No.: US 2020/0372983 A1
(43) Pub. Date: Nov. 26, 2020

(54) METHODS AND DEVICES FOR STORING AND PROCESSING ELECTRONIC MEDICAL RECORD ON BLOCKCHAIN

(71) Applicant: Alibaba Group Holding Limited, George Town (KY)

(72) Inventors: Yixiang ZHANG, Hangzhou (CN); Xueqing YANG, Hangzhou (CN); Shubo LI, Hangzhou (CN); Zhihua LIANG, Hangzhou (CN)

(73) Assignee: Alibaba Group Holding Limited

(21) Appl. No.: 16/775,869

(22) Filed: Jan. 29, 2020

Related U.S. Application Data
(63) Continuation of application No. PCT/CN2019/087745, filed on May 21, 2019.

(51) Int. Cl. G16H 10/60; G16H 20/10

(52) U.S. Cl. CPC G16H 10/60; G16H 20/10

(57) ABSTRACT

Disclosed herein are methods, devices, and apparatuses, including computer programs stored on computer-readable media, for storing and processing electronic medical records. One of the methods includes: receiving, by a node in a blockchain system, an electronic medical record for a patient; incorporating the electronic medical record into a blockchain maintained by the blockchain system; determining, by the node, whether the electronic medical record contains a prescription; and in response to a determination that the electronic medical record contains a prescription, notifying the patient of the prescription.

### TECHNICAL FIELD

The specification relates generally to computer technologies, and more particularly, to methods and devices for storing and processing electronic medical record on blockchain.

### BACKGROUND

Electronic medical records may contain electronically stored medical and health information regarding patients. Electronic medical records may be collected by clinicians, physicians, nurses, or other staff members at clinics or hospitals. Electronic medical records may include data such as demographics, medical histories, medication and allergy information, immunization status, laboratory test results, radiology images, vital signs, personal statistics like age, weight, and medical prescriptions. Electronic medical records can be shared through network-connected information sharing platforms, which may be utilized by clinicians to assist diagnoses of patients and by pharmacists to fill prescriptions for patients.

There are several information sharing platforms currently available. These information sharing platforms do not prescribe to any standard, making it difficult for them to provide cross-platform communications. For example, a pharmacist using one information sharing platform may have difficulties retrieving or verifying a prescription for a patient whose clinician uses a different information sharing platform. Furthermore, because electronic medical records can be copied, it creates possibilities for patients to make unauthorized copies of their prescriptions. Therefore, there is a need for a method to improve the processing and handling of electronic medical records.

### SUMMARY

In one aspect, a computer-implemented method for storing and processing electronic medical records includes: receiving, by a node in a blockchain system, an electronic medical record for a patient; incorporating the electronic medical record into a blockchain maintained by the blockchain system; determining, by the node, whether the electronic medical record contains a prescription; and in response to a determination that the electronic medical record contains a prescription, notifying the patient of the prescription.

In another aspect, a device for storing and processing electronic medical records includes: one or more processors; and one or more computer-readable memories coupled to the one or more processors and having instructions stored thereon that are executable by the one or more processors to receive an electronic medical record for a patient; incorporate the electronic medical record into a blockchain maintained by a blockchain system; determine whether the electronic medical record contains a prescription; and in response to a determination that the electronic medical record contains a prescription, notify the patient of the prescription.

In still another aspect, a non-transitory computer-readable medium have stored therein instructions that, when executed by a processor of a device, cause the device to perform a method for storing and processing electronic medical records. The method includes: receiving, by a node in a blockchain system, an electronic medical record for a patient; incorporating the electronic medical record into a blockchain maintained by the blockchain system; determining, by the node, whether the electronic medical record contains a prescription; and in response to a determination that the electronic medical record contains a prescription, notifying the patient of the prescription.

### BRIEF DESCRIPTION OF THE DRAWINGS

The accompanying drawings, which are incorporated in and constitute a part of this specification, illustrate embodiments. In the following description, which refers to the drawings, the same numbers in different drawings represent the same or similar elements unless otherwise represented.

FIG. 1 is a schematic diagram of a blockchain system, according to an embodiment.

FIG. 2 is a schematic diagram of a computing device for implementing a node in a blockchain system, according to an embodiment.

FIG. 3 is a flow chart of a method for storing and processing an electronic medical record on a blockchain, according an embodiment.

FIG. 4 is a flow chart of a method for storing and processing an electronic medical record on a blockchain, according an embodiment.

FIG. 5 is a flow chart of a method for storing and processing an electronic medical record on a blockchain, according an embodiment.

FIG. 6 is a flow chart of a method for storing and processing an electronic medical record on a blockchain, according an embodiment.

FIG. 7 is a block diagram of an apparatus for storing and processing an electronic medical record on a blockchain, according to an embodiment.

### DETAILED DESCRIPTION

Embodiments of the specification provide methods and devices for storing and processing electronic medical records. The methods and devices utilize blockchain systems to store electronic medical records, enabling participating entities, e.g., hospitals, pharmacies, etc., to store electronic medical records securely and immutably. The methods and devices also support executions of smart contracts on blockchain systems, enabling participating entities to use smart contracts to automatically invalidate prescriptions once the prescriptions are filled.

Embodiments disclosed in the specification have one or more technical effects. In some embodiments, the methods and devices record electronic medical records on a blockchain system. This allows for better communications between various entities using the record electronic medical records. This also provides a patient seeking medical care at a first hospital the ability to go to a second hospital for further consultation, where the second hospital can retrieve the patient's electronic medical records securely from the blockchain system. In some embodiments, the methods and devices also support executions of smart contracts on the blockchain system. This allows participating entities to use smart contracts to automatically invalidate prescriptions once the prescriptions are filled, preventing the possibilities of patients creating unauthorized copies of their prescriptions. This also provides the blockchain system with the abilities to detect and reject unauthorized prescriptions, further enhancing patient safety.

Blockchain systems, also known as distributed ledger systems (DLSs) or consensus systems, may include any DLSs, without referencing any particular use case, and may be used for public, private, and consortium blockchain networks. A blockchain system may be implemented using a peer-to-peer (P2P) network, in which the nodes communicate directly with each other, e.g., without the need of a fixed, central server. Each node in the P2P network may initiate communication with another node in the P2P network.

A blockchain system maintains one or more blockchains. A blockchain is a data structure that stores data, e.g., transactions, in a way that may prevent tampering and manipulation of the data by malicious parties. The transactions stored in this manner may be immutable and subsequently verified. A blockchain includes one or more blocks. Each block is linked to a previous block immediately before it in the blockchain by including a cryptographic hash of the previous block. Each block also may include a timestamp, its own cryptographic hash, and one or more transactions. The transactions, which generally have already been verified by the nodes of the blockchain system, may be hashed and encoded into a data structure, such as a Merkle tree. In a Merkle tree, data at leaf nodes of the tree is hashed, and all hashes in each branch of the tree may be concatenated at a root of the branch. This process continues up the tree to the root of the entire tree, which stores a hash that is representative of all data in the tree. A hash purporting to be of a transaction stored in the tree can be quickly verified by determining whether it is consistent with the structure of the tree.

A blockchain system includes a network of computing nodes that manage, update, and maintain one or more blockchains. The network may be a public blockchain network, a private blockchain network, or a consortium blockchain network. For example, numerous entities, such as hundreds, thousands, or even millions of entities, can operate in a public blockchain network, and each of the entities operates at least one node in the public blockchain network. Accordingly, the public blockchain network can be considered a public network with respect to the participating entities. Sometimes, a majority of entities (nodes) must sign every block for the block to be valid and added to the blockchain of the blockchain network. Examples of public blockchain networks include particular peer-to-peer payment networks that leverage a distributed ledger, referred to as blockchain.

In general, a public blockchain network may support public transactions. A public transaction is shared with all of the nodes in the public blockchain network, and is stored in a global blockchain. A global blockchain is a blockchain replicated across all nodes, and all nodes are in perfect state consensus with respect to the global blockchain. To achieve consensus (e.g., agreement to the addition of a block to a blockchain), a consensus protocol is implemented in the public blockchain network. Examples of consensus protocols include proof-of-work (POW) (e.g., implemented in the some crypto-currency networks), proof-of-stake (POS), and proof-of-authority (POA).

In general, a private blockchain network may be provided for a particular entity, which centrally controls read and write permissions. The entity controls which nodes are able to participate in the blockchain network. Consequently, private blockchain networks are generally referred to as permissioned networks that place restrictions on who is allowed to participate in the network, and on their level of participation (e.g., only in certain transactions). Various types of access control mechanisms can be used (e.g., existing participants vote on adding new entities, a regulatory authority can control admission).

In general, a consortium blockchain network may be private among the participating entities. In a consortium blockchain network, the consensus process is controlled by an authorized set of nodes, one or more nodes being operated by a respective entity (e.g., a financial institution, insurance company). For example, a consortium of ten (10) entities (e.g., financial institutions, insurance companies) can operate a consortium blockchain network, each of which operates at least one node in the consortium blockchain network. Accordingly, the consortium blockchain network can be considered a private network with respect to the participating entities. In some examples, each entity (node) must sign every block in order for the block to be valid, and added to the blockchain. In some examples, at least a sub-set of entities (nodes) (e.g., at least 7 entities) must sign every block in order for the block to be valid, and added to the blockchain.

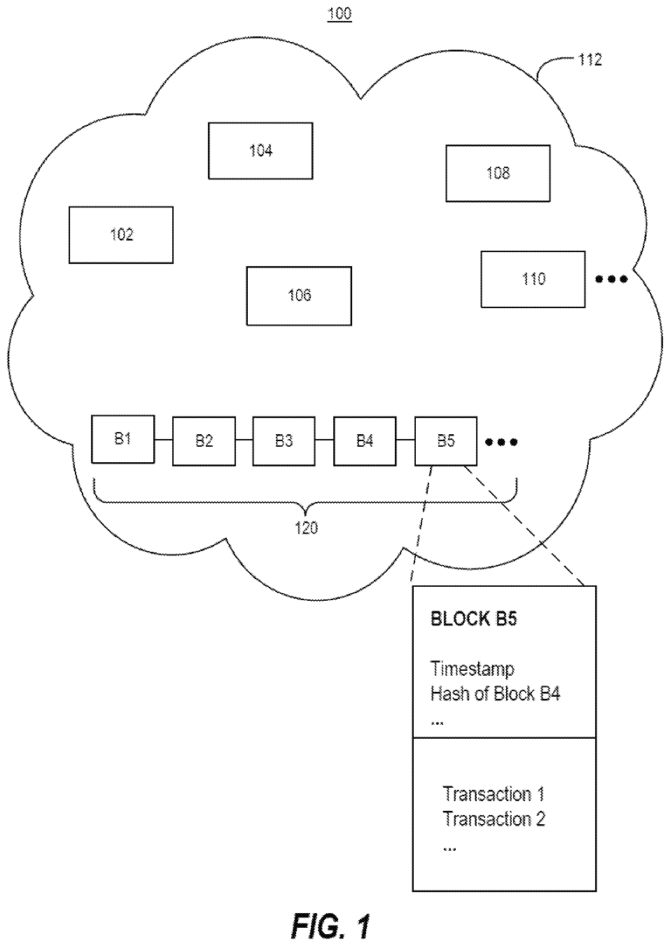

FIG. 1 illustrates a schematic diagram of a blockchain system 100, according to an embodiment. Referring to FIG. 1, the blockchain system 100 may include a plurality of nodes, e.g., nodes 102-110, configured to operate on a blockchain 120. The nodes 102-110 may form a network 112, such as a peer-to-peer (P2P) network. Each of the nodes 102-110 may be a computing device, such as a computer or a computer system, configured to store a copy of the blockchain 120, or may be software running on the computing device, such as a process or an application. Each of the nodes 102-110 may have a unique identifier.

The blockchain 120 may include a growing list of records in the form of data blocks, such as blocks B1-B5 in FIG. 1. Each of the blocks B1-B5 may include a timestamp, a cryptographic hash of a previous block, and data of the present block, which may be transactions such as monetary transactions. For example, as illustrated in FIG. 1, block B5 may include a timestamp, a cryptographic hash of block B4, and transaction data of block B5. Also, for example, a hashing operation may be performed on the previous block to generate the cryptographic hash of the previous block. The hashing operation may convert inputs of various lengths into cryptographic outputs of a fixed length through a hash algorithm, such as SHA-256.

The nodes 102-110 may be configured to perform an operation on the blockchain 120. For example, when a node, e.g., the node 102, wants to store new data onto the blockchain 120, that node may generate a new block to be added to the blockchain 120 and broadcast the new block to other nodes, e.g., the nodes 104-110, in the network 112. Based on legitimacy of the new block, e.g., validity of its signature and transactions, the other nodes may determine to accept the new block, such that the node 102 and the other nodes may add the new block to their respective copies of the blockchain 120. As this process repeats, more and more blocks of data may be added to the blockchain 120.

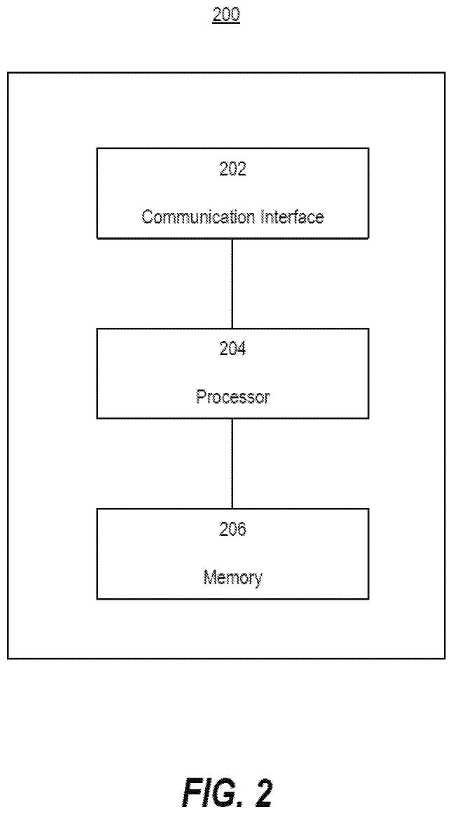

FIG. 2 illustrates a schematic diagram of a computing device 200 for implementing a node, e.g., the node 102 (FIG. 1), in a blockchain system, according to an embodiment. Referring to FIG. 2, the computing device 200 may include a communication interface 202, a processor 204, and a memory 206.

The communication interface 202 may facilitate communications between the computing device 200 and devices implementing other nodes, e.g., nodes 104-110 (FIG. 1), in the network. In some embodiments, the communication interface 202 is configured to support one or more communication standards, such as an Internet standard or protocol, an Integrated Services Digital Network (ISDN) standard, etc. In some embodiments, the communication interface 202 may include one or more of a Local Area Network (LAN) card, a cable modem, a satellite modem, a data bus, a cable, a wireless communication channel, a radio-based communication channel, a cellular communication channel, an Internet Protocol (IP) based communication device, or other communication devices for wired and/or wireless communications. In some embodiments, the communication interface 202 may be based on public cloud infrastructure, private cloud infrastructure, hybrid public/private cloud infrastructure.

The processor 204 may include one or more dedicated processing units, application-specific integrated circuits (ASICs), field-programmable gate arrays (FPGAs), or various other types of processors or processing units. The processor 204 is coupled with the memory 206 and is configured to execute instructions stored in the memory 206.

The memory 206 may store processor-executable instructions and data, such as a copy of the blockchain 120 (FIG. 1). The memory 206 may include any type of volatile or non-volatile memory devices, or a combination thereof, such as a static random-access memory (SRAM), an electrically erasable programmable read-only memory (EEPROM), an erasable programmable read-only memory (EPROM), a programmable read-only memory (PROM), a read-only memory (ROM), a magnetic memory, a flash memory, or a magnetic or optical disk. When the instructions in the memory 206 are executed by the processor 204, the computing device 200 may perform an operation on the blockchain 120.

Referring back to FIG. 1. In some embodiments, the blockchain system 100 may be implemented to support various types of participating entities, or users, including, e.g., patients, hospitals, pharmacies, and the like. The various users may utilize the blockchain system 100 to retrieve or record information, including, e.g., electronic medical record information.

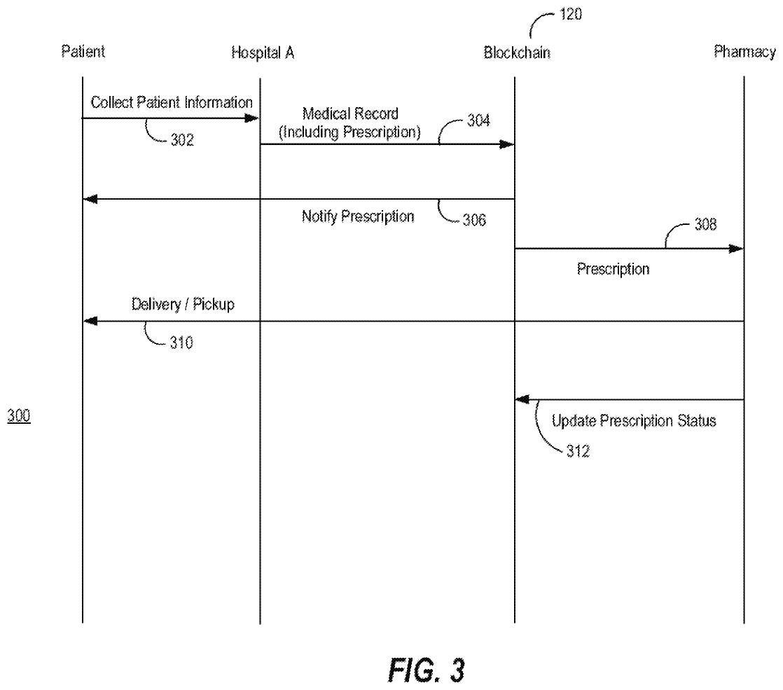

FIG. 3 illustrates a flow chart of a method 300 for storing and processing an electronic medical record on a blockchain, e.g., the blockchain 120 (FIG. 1), according to an embodiment.

At step 302, a Patient may visit a Hospital A seeking medical care. The Hospital A may collect information from the Patient, including, e.g., symptoms, age, weight, demographic information, medical history, medication and allergy information, immunization status, laboratory test results, radiology images, vital signs, or the like. The Hospital A may make a diagnosis based on the information collected and prescribe certain medications to the Patient.

At step 304, the Hospital A may submit the diagnosis, the prescription, and the information collected as a part of a medical record for the Patient to be recorded on the blockchain 120. The nodes 102-110 of the blockchain system 100 (FIG. 1) that maintains the blockchain 120 may receive the medical record and incorporate the medical record into a block to be added to the blockchain 120. In an embodiment, similar to the description above in connection with FIG. 1, the node 102 includes data corresponding to the medical record in a new block, and broadcasts the new block in the blockchain system. The node 102 further determines whether the new block is accepted in the blockchain system, and in response to a determination that the new block is accepted in the blockchain system, adds the new block onto the blockchain, e.g., by adding the new block onto a copy of the blockchain maintained by the node 102.

At step 306, the blockchain system 100 may notify the Patient of the receipt of the diagnosis and the prescription from Hospital A. In some embodiments, the blockchain system 100 may provide an identifier to the Patient, allowing the Patient to properly identify the diagnosis and the prescription using the identifier. In some embodiments, the identifier may be alphanumeric. In some embodiments, the identifier may include an address where the diagnosis and prescription information is stored. In some embodiments, the blockchain system 100 may notify the Patient through an account the Patient has with the blockchain system 100. In some embodiments, the blockchain system 100 may notify the Patient via other communication means, including, e.g., text messages, electronic mails, telephone calls, and the like. The Patient may decide which pharmacy to visit to fill the prescription. In some embodiments, the Patient may present the prescription to a chosen Pharmacy and wait to pick up the medications prescribed in the prescription. In some embodiments, the Patient may request the Pharmacy to retrieve the prescription from the blockchain 120, e.g., using the identifier, and fill the prescription based on the retrieved prescription. The Patient may pick up the medications at the Pharmacy or request the Pharmacy to deliver the medications to a specified location.

At step 308, the Pharmacy may retrieve the prescription from the blockchain 120, e.g., using the identifier or the Patient's identifying information. In some embodiments, the Pharmacy may retrieve the prescription from the blockchain 120 even if the Pharmacy has already received a purported prescription from the Patient. In this manner, the Pharmacy may be able to verify the accuracy of the purported prescription received from the Patient.

At step 310, the Pharmacy may fill the prescription based on the prescription retrieved from the blockchain 120. In some embodiments, the Pharmacy may only fill the prescription based on the prescription retrieved from the blockchain 120, regardless of what the purported prescription received from the Patient shows. The Pharmacy may then deliver the medications prescribed in the prescription to the Patient or allow the Patient to pick up the medications if the Patient chooses to do so.

At step 312, the Pharmacy may update a status associated with the prescription recorded on the blockchain 120. For example, the Pharmacy may record information regarding a type and an amount of medications delivered to or picked up by the Patient. The information provided by the Pharmacy may be incorporated into the blockchain 120, which may then be utilized by one or more smart contracts executing on the blockchain system 100 to update the prescription recorded on the blockchain 120.

Smart contracts are computer protocols implemented in the form of computer code that are incorporated into the blockchain 120, to facilitate, verify, or enforce the negotiation or performance of contracts. For example, a user of the blockchain system 100 may program agreed terms into a smart contract using a programming language, such as C++, Java, Solidity, Python, etc., and when the terms are met, the smart contract may be automatically executed by the blockchain system 100, e.g., to perform a transaction. Also for example, the smart contract may include a plurality of subroutines or functions, each of which may be a sequence of program instructions that performs a specific task. The smart contract may be operational code that is fully or partially executed without human interaction.

In some embodiments, a smart contract may be incorporated into the blockchain 120 to verify and update validities of the prescriptions recorded on the blockchain 120. When the Pharmacy updates the status associated with a particular prescription recorded on the blockchain 120, the smart contract may execute automatically to verify and update that particular prescription based on the type and the amount of medications reported by the Pharmacy as having been delivered to (or picked up by) the Patient. If the prescription has been filled completely and no refill prescription remains, the smart contract may invalidate the prescription to prevent the prescription from being used by the Patient again. If, on the other hand, the prescription has not been filled completely or refill is still available, the smart contract may update the prescription to indicate the remaining amount, allowing the steps 308-312 to be repeated until the prescription is filled completely.

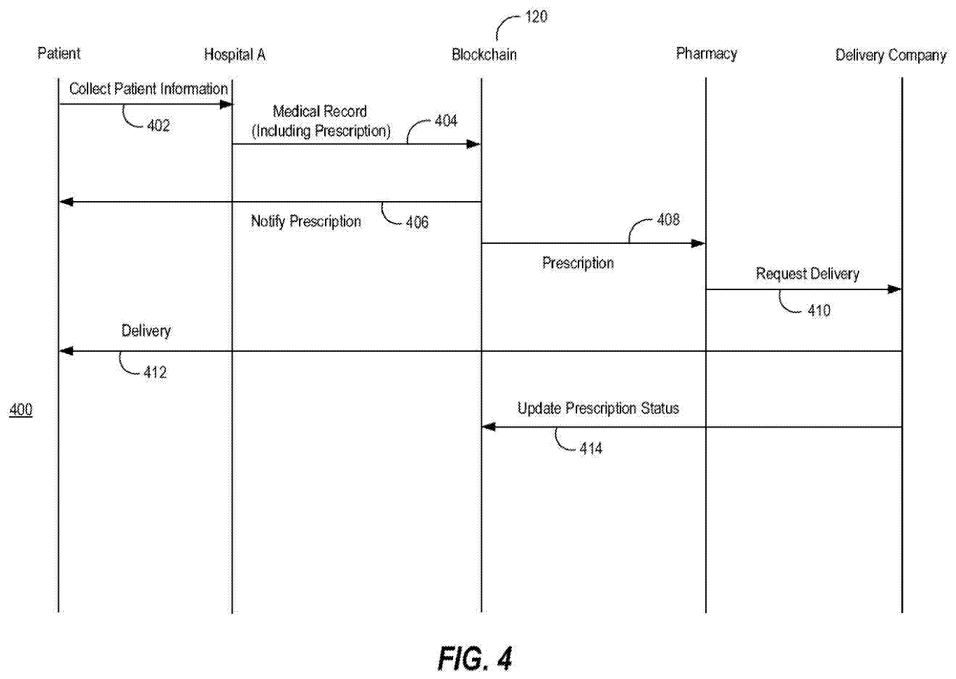

In some embodiments, the Pharmacy may outsource the delivery service to a third-party provider. In some embodiments, the third-party provider may also utilize the blockchain 120 to store and process electronic medical records in manners similar to that described above. FIG. 4 illustrates a flow chart of a method 400 for storing and processing an electronic medical record on a blockchain, e.g., the blockchain 120 (FIG. 1), according to an embodiment.

At step 402, a Patient may visit a Hospital A seeking medical care. The Hospital A may collect information from the Patient. The Hospital A may make a diagnosis based on the information collected and prescribe certain medications to the Patient.

At step 404, the Hospital A may submit the diagnosis, the prescription, and the information collected as a part of a medical record for the Patient to be recorded on the blockchain 120. The nodes 102-110 of the blockchain system 100 (FIG. 1) that maintains the blockchain 120 may receive the medical record and incorporate the medical record into a block to be added to the blockchain 120. In an embodiment, similar to the description above in connection with FIG. 1, the node 102 includes data corresponding to the medical record in a new block, and broadcasts the new block in the blockchain system. The node 102 further determines whether the new block is accepted in the blockchain system, and in response to a determination that the new block is accepted in the blockchain system, adds the new block onto the blockchain, e.g., by adding the new block onto a copy of the blockchain maintained by the node 102.

At step 406, the blockchain system 100 may notify the Patient of the receipt of the diagnosis and the prescription from Hospital A. The Patient may decide which Pharmacy to visit to fill the prescription.

At step 408, the Pharmacy chosen by the Patient may retrieve the prescription from the blockchain 120 and fill the prescription accordingly.

At step 410, the Pharmacy may request a third-party provider, e.g., a Delivery Company, to deliver the medications prescribed in the prescription to the Patient.

At step 412, the Delivery Company may deliver the medications to the Patient as requested.

At step 414, the Delivery Company may update the status associated with the prescription recorded on the blockchain 120. For example, in some embodiments, the Delivery Company may record information regarding the type and the amount of medications the Delivery Company picked up from the Pharmacy, along with a timestamp. The Delivery Company may also record tracking information regarding the delivery status. The Delivery Company may further record information confirming the delivery of the medications, including information regarding the type and the amount of medications delivered, along with a timestamp.

The information provided by the Delivery Company may be incorporated into the blockchain 120, allowing one or more smart contracts executing on the blockchain system 100 to update the prescription recorded on the blockchain 120. If the prescription has been filled completely and no refill prescription remains, a smart contract may invalidate the prescription to prevent the prescription from being used by the Patient again. If, on the other hand, the prescription has not been filled completely or refill is still available, the smart contract may update the prescription to indicate the remaining amount, allowing the steps 408-414 to be repeated until the prescription is filled completely.

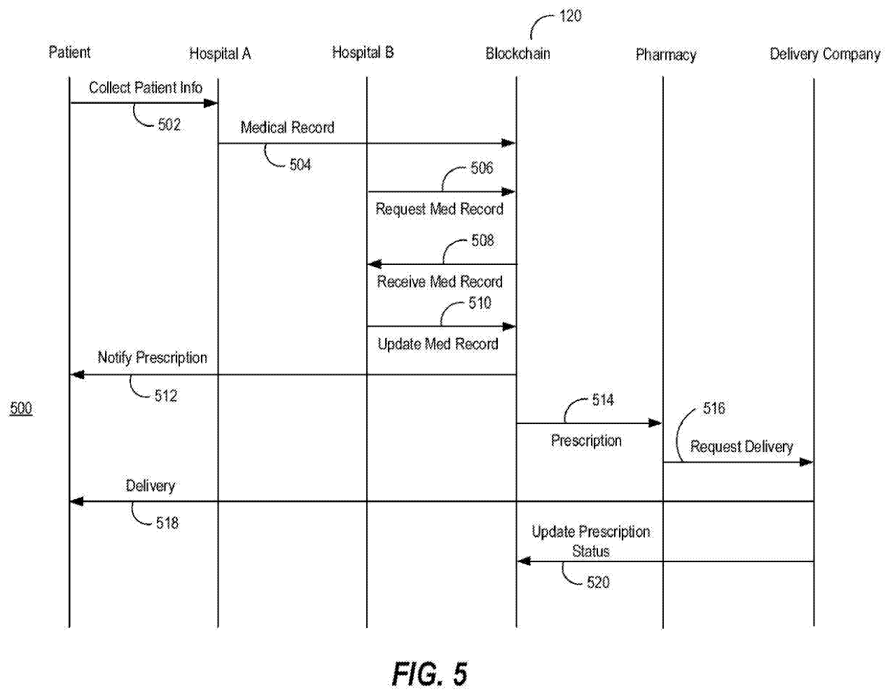

In some embodiments, two or more hospitals may jointly participate in a diagnosis process. Two or more hospitals may utilize the blockchain 120 to store and process electronic medical records in manners similar to that described above. FIG. 5 illustrates a flow chart of a method 500 for storing and processing an electronic medical record on a blockchain, e.g., the blockchain 120 (FIG. 1), according to an embodiment.

At step 502, a Patient may visit a Hospital A seeking medical care. The Hospital A may collect information from the Patient. The Hospital A may make a preliminary diagnosis based on the information collected.

At step 504, the Hospital A may submit the preliminary diagnosis and the information collected as a part of a medical record for the Patient to be recorded on the blockchain 120. The nodes 102-110 of the blockchain system 100 (FIG. 1) that maintains the blockchain 120 may receive the medical record and incorporate the medical record into a new block to be added to the blockchain 120. In an embodiment, similar to the description above in connection with FIG. 1, the node 102 includes data corresponding to the medical record in a new block, and broadcasts the new block in the blockchain system. The node 102 further determines whether the new block is accepted in the blockchain system, and in response to a determination that the new block is accepted in the blockchain system, adds the new block onto the blockchain, e.g., by adding the new block onto a copy of the blockchain maintained by the node 102.

At step 506, a Hospital B may join the diagnosis process, e.g., at the request of the Patient or the Hospital A. The Hospital B may request the medical record for the Patient from the blockchain 120.

At step 508, the Hospital B may receive the medical record for the Patient from the blockchain 120. The Hospital B may collect additional information, if needed, from the Patient, including, e.g., additional laboratory test results, radiology images, vital signs, and the like. The Hospital B may make a second diagnosis. The Hospital B may also prescribe certain medications to the Patient after the second diagnosis.

At step 510, the Hospital B may submit the second diagnosis, the prescription, and the information collected as a part of the updated medical record for the Patient to be recorded on the blockchain 120. The nodes 102-110 of the blockchain system 100 (FIG. 1) that maintains the blockchain 120 may receive the updated medical record and incorporate the updated medical record into a block to be added to the blockchain 120, similar to step 504.

At step 512, the blockchain system 100 may notify the Patient of the receipt of the second diagnosis and the prescription from Hospital B. The Patient may decide which Pharmacy to visit to fill the prescription.

At step 514, the Pharmacy chosen by the Patient may retrieve the prescription from the blockchain 120 and fill the prescription accordingly.

At step 516, the Pharmacy may request a third-party provider, e.g., a Delivery Company, to deliver the medications prescribed in the prescription to the Patient. Alternatively and/or additionally, the Pharmacy may deliver the medications itself, or allow the Patient to pick up the medications at the Pharmacy.

If the Pharmacy requested delivery by the Delivery Company, the Delivery Company, at step 518, may deliver the medications to the Patient. At step 520, the Delivery Company may update the status associated with the prescription recorded on the blockchain 120.

Alternatively, if the Pharmacy does not utilize third-party providers, the Pharmacy may deliver the medications to the Patient or allow the Patient to pick up the medications at the Pharmacy. The Pharmacy may then update the status associated with the prescription recorded on the blockchain 120.

The information provided by the Delivery Company or the Pharmacy for updating the status may be incorporated into the blockchain 120, which may then be utilized by one or more smart contracts executing on the blockchain system 100 to update the prescription recorded on the blockchain 120. If the prescription has been filled completely and no refill prescription remains, a smart contract may invalidate the prescription to prevent the prescription from being used by the Patient again. If, on the other hand, the prescription has not been filled completely or refill is still available, the smart contract may update the prescription to indicate the remaining amount, allowing the steps 514-520 to be repeated until the prescription is filled completely.

It is to be understood that other hospitals and participating entities, including, e.g., pharmacies and delivery companies, may utilize the blockchain 120 to store and process electronic medical records in manners similar to that described above. It is also to be understood that while the examples above depicted patients, hospitals, pharmacies, and delivery companies as users of the blockchain system 100, such depictions are not meant to be limiting.

In some embodiments, the blockchain system 100 may implement a consortium blockchain network that is private among the participating entities (e.g., hospitals, pharmacies, and delivery companies). The nodes 102-110 (FIG. 1) forming the consortium blockchain network may be operated by the participating entities. In some embodiments, each participating entity may participate in the blockchain system 100 under its respective account. For example, the Hospital A may participate in the blockchain system 100 under a first account, which is different from a second account utilized by the Hospital B. In some embodiments, the blockchain system 100 may allow hospitals, pharmacies, and delivery companies to implement policies regarding who should be allowed to act on the blockchain system 100 on their behalf. In some embodiments, the blockchain system 100 may allow individuals such as clinicians, physicians, nurses, pharmacists, or other staff members to hold individual accounts and act on the blockchain system 100 individually.

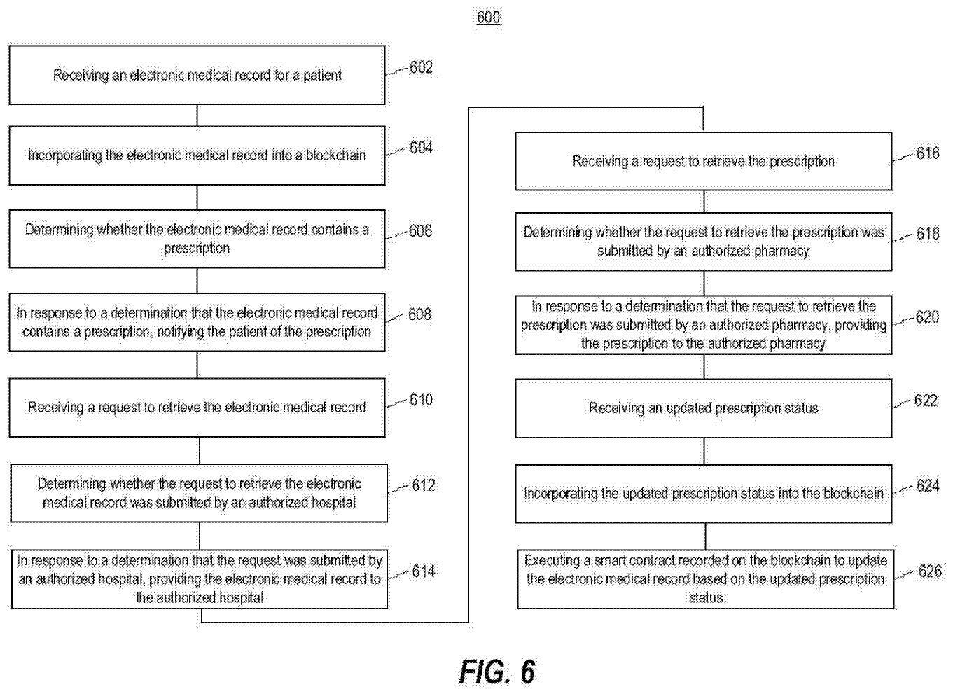

FIG. 6 illustrates a flow chart of a method 600 for storing and processing electronic medical records on a blockchain, e.g., the blockchain 120 (FIG. 1), according to an embodiment. The method 600 may be performed by one or more nodes in a blockchain system that maintains the blockchain, e.g., the nodes 102-110 in the blockchain system 100 (FIG. 1).

At step 602, a node, e.g., the node 102, may receive an electronic medical record for a patient. The electronic medical record may include information collected from the patient, such as symptoms, age, weight, demographic information, medical history, medication and allergy information, immunization status, laboratory test results, radiology images, vital signs, or the like. The electronic medical record may also include a diagnosis and a prescription.

At step 604, the node 102 may incorporate the electronic medical record into the blockchain 120. At step 606, the node 102 may determine whether the electronic medical record contains a prescription. At step 608, in response to a determination that the electronic medical record contains a prescription, the node 102 may notify the patient of the prescription.

In some embodiments, the method 600 may allow two or more hospitals to jointly participate in a diagnosis process. For example, at step 610, the node 102 may receive a request to retrieve the patient's electronic medical record. At step 612, the node 102 may determine whether the request was submitted by an authorized hospital. In some embodiments, only authorized hospitals, e.g., hospitals having setup valid accounts with the blockchain system 100 and authorized by the patient, may request to retrieve the patient's electronic medical record. At step 614, in response to a determination that the request was submitted by an authorized hospital, the node 102 may provide the electronic medical record to the authorized hospital. The node 102 may also receive, from the authorized hospital, an updated electronic medical record. The node may incorporate the updated electronic medical record into the blockchain. The node 102 may further determine whether the updated electronic medical record contains a prescription, and if so, the node 102 may notify the patient of the prescription.

The patient, upon receiving the notification, may decide which pharmacy to visit to fill the prescription. In some embodiments, the pharmacy chosen by the patient may be required to retrieve the prescription from the blockchain 120. For example, at step 616, the node 102 may receive a request to retrieve the prescription. At step 618, the node 102 may determine whether the request to retrieve the prescription was submitted by an authorized pharmacy. In some embodiments, only authorized pharmacies, e.g., pharmacies having setup valid accounts with the blockchain system 100, may request to retrieve prescriptions. At step 620, in response to a determination that the request to retrieve the prescription was submitted by an authorized pharmacy, the node 102 may provide the prescription to the authorized pharmacy.

In some embodiments, the pharmacy chosen by the patient may be responsible for updating the status of the prescription. If the pharmacy utilizes a third-party provider, e.g., a delivery company, to handle the delivery of the medications prescribed in the prescription, the third-party provider may also update the status of the prescription. At step 622, the node 102 may receive an updated prescription status. At step 624, the node 102 may incorporate the updated prescription status into the blockchain 120. At step 626, the node 102 may execute a smart contract recorded on the blockchain 120 to update the electronic medical record based on the updated prescription status. The smart contract may invalidate the prescription contained in the electronic medical record when the updated prescription status indicates the prescription contained in the electronic medical record has been filled completely.

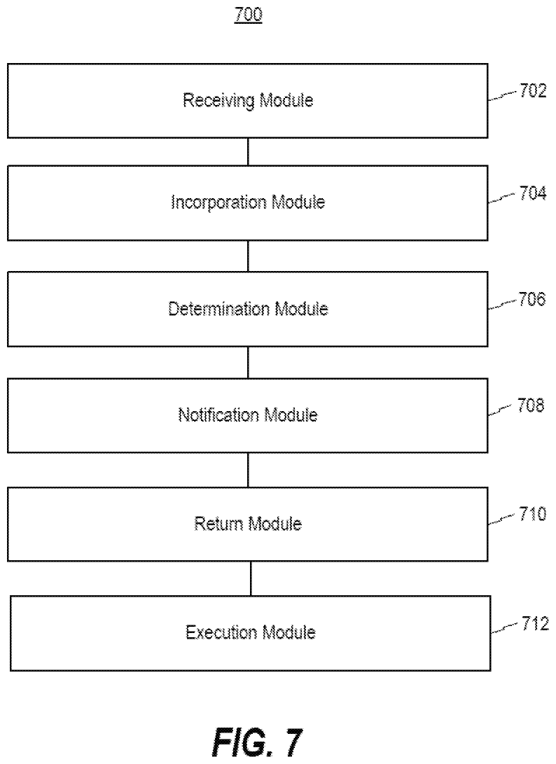

FIG. 7 is a block diagram of an apparatus 700 for storing and processing electronic medical records on a blockchain, according to an embodiment. For example, the apparatus 700 may be an implementation of a software process, and may correspond to the method 600 (FIG. 6). Referring to FIG. 7, the apparatus 700 may include a receiving module 702, an incorporation module 704, a determination module 706, a notification module 708, a return module 710, and an execution module 712.

The receiving module 702 may receive an electronic medical record for a patient. The incorporation module 704 may incorporate the electronic medical record into a blockchain, e.g., the blockchain 120 (FIG. 1). The determination module 706 may determine whether the electronic medical record contains a prescription. The notification module 708 may notify the patient of the prescription if the electronic medical record contains a prescription.

In some embodiments, the apparatus 700 may allow two or more hospitals to jointly participate in a diagnosis process. In such embodiments, the receiving module 702 may receive a request to retrieve the patient's electronic medical record. The determination module 706 may determine whether the request was submitted by an authorized hospital. In response to a determination that the request was submitted by an authorized hospital, the return module 710 may provide the electronic medical record to the authorized hospital. In some embodiments, the receiving module 702 may further receive, from the authorized hospital, an updated electronic medical record. The incorporation module 704 may incorporate the updated electronic medical record into the blockchain 120. The determination module 706 may further determine whether the updated electronic medical record contains a prescription, and if so, the notification module 708 may notify the patient of the prescription.

In some embodiments, the apparatus 700 may also allow a pharmacy chosen by the patient to retrieve the prescription from the blockchain 120. In such embodiments, the receiving module 702 may receive a request to retrieve the prescription. The determination module 706 may determine whether the request to retrieve the prescription was submitted by an authorized pharmacy. In response to a determination that the request to retrieve the prescription was submitted by an authorized pharmacy, the return module 710 may provide the prescription to the authorized pharmacy.

In some embodiments, the pharmacy chosen by the patient (or a third-party provider utilized by the pharmacy) may be required to update the status of the prescription. In such embodiments, the receiving module 702 may receive an updated prescription status. The incorporation module 704 may incorporate the updated prescription status into the blockchain 120. The execution module 712 may execute a smart contract recorded on the blockchain 120 to update the electronic medical record based on the updated prescription status. The smart contract may invalidate the prescription contained in the electronic medical record when the updated prescription status indicates the prescription contained in the electronic medical record has been filled completely.

Each of the above described modules may be implemented as software, or hardware, or a combination of software and hardware. For example, each of the above described modules may be implemented using a processor executing instructions stored in a memory. Also, for example, each the above described modules may be implemented with one or more application specific integrated circuits (ASICs), digital signal processors (DSPs), digital signal processing devices (DSPDs), programmable logic devices (PLDs), field programmable gate arrays (FPGAs), controllers, micro-controllers, microprocessors, or other electronic components, for performing the described methods. Further for example, each of the above described modules may be implemented by using a computer chip or an entity, or implemented by using a product having a certain function. In one embodiment, the apparatus 700 may be a computer, and the computer may be a personal computer, a laptop computer, a cellular phone, a camera phone, a smartphone, a personal digital assistant, a media player, a navigation device, an email receiving and sending device, a game console, a tablet computer, a wearable device, or any combination of these devices.

For an implementation process of functions and roles of each module in the apparatus 700, references can be made to corresponding steps in the above-described methods. Details are omitted here for simplicity.

In some embodiments, a computer program product may include a non-transitory computer-readable storage medium having computer-readable program instructions thereon for causing a processor to carry out the above-described methods.

The computer-readable storage medium may be a tangible device that can store instructions for use by an instruction execution device. The computer-readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer-readable storage medium includes the following: a portable computer diskette, a hard disk, a random access memory (RAM), a read-only memory (ROM), an erasable programmable read-only memory (EPROM), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing.

The computer-readable program instructions for carrying out the above-described methods may be assembler instructions, instruction-set-architecture (ISA) instructions, machine instructions, machine dependent instructions, microcode, firmware instructions, state-setting data, or source code or object code written in any combination of one or more programming languages, including an object oriented programming language, and conventional procedural programming languages. The computer-readable program instructions may execute entirely on a computing device as a stand-alone software package, or partly on a first computing device and partly on a second computing device remote from the first computing device. In the latter scenario, the second, remote computing device may be connected to the first computing device through any type of network, including a local area network (LAN) or a wide area network (WAN).

The computer-readable program instructions may be provided to a processor of a general-purpose or special-purpose computer, or other programmable data processing apparatus to produce a machine, such that the instructions, which execute via the processor of the computer or other programmable data processing apparatus, create means for implementing the above-described methods.

The flow charts and diagrams in the figures illustrate the architecture, functionality, and operation of possible implementations of devices, methods, and computer program products according to various embodiments of the specification. In this regard, a block in the flow charts or diagrams may represent a software program, segment, or portion of code, which comprises one or more executable instructions for implementing specific functions. It should also be noted that, in some alternative implementations, the functions noted in the blocks may occur out of the order noted in the figures. For example, two blocks shown in succession may, in fact, be executed substantially concurrently, or the blocks may sometimes be executed in the reverse order, depending upon the functionality involved. It will also be noted that each block of the diagrams and/or flow charts, and combinations of blocks in the diagrams and flow charts, may be implemented by special purpose hardware-based systems that perform the specified functions or acts, or combinations of special purpose hardware and computer instructions.

It is appreciated that certain features of the specification, which are, for clarity, described in the context of separate embodiments, may also be provided in combination in a single embodiment. Conversely, various features of the specification, which are, for brevity, described in the context of a single embodiment, may also be provided separately or in any suitable subcombination or as suitable in any other described embodiment of the specification. Certain features described in the context of various embodiments are not essential features of those embodiments, unless noted as such.

Although the specification has been described in conjunction with specific embodiments, many alternatives, modifications and variations will be apparent to those skilled in the art. Accordingly, the following claims embrace all such alternatives, modifications and variations that fall within the terms of the claims.

1. A computer-implemented method for storing and processing electronic medical records, the method comprising:
receiving, by a node in a blockchain system, an electronic medical record for a patient;
incorporating the electronic medical record into a blockchain maintained by the blockchain system, the incorporating comprising:
including data corresponding to the electronic medical record in a data block;
broadcasting the data block in the blockchain system;
determining whether the data block is accepted in the blockchain system; and
in response to a determination that the data block is accepted in the blockchain system, adding the data block to the blockchain;
determining, by the node, whether the electronic medical record contains a prescription;
in response to a determination that the electronic medical record contains a prescription, notifying the patient of the prescription;
receiving an updated prescription status;
incorporating the updated prescription status into the blockchain; and
executing a smart contract recorded on the blockchain to update the electronic medical record based on the updated prescription status to indicate whether refill of the prescription is available.

2. The method of claim 1, further comprising:
receiving, by the node, a request to retrieve the prescription;
determining, by the node, whether the request to retrieve the prescription was submitted by an authorized pharmacy; and
in response to a determination that the request to retrieve the prescription was submitted by an authorized pharmacy, providing the prescription to the authorized pharmacy.

3. (canceled)

4. (canceled)

5. The method of claim 1, wherein the smart contract invalidates the prescription contained in the electronic medical record when the updated prescription status indicates the prescription contained in the electronic medical record has been filled completely and no refill prescription remains.

6. The method of claim 1, further comprising:
receiving, by the node, a request to retrieve the electronic medical record;
determining, by the node, whether the request to retrieve the electronic medical record was submitted by an authorized hospital; and
in response to a determination that the request to retrieve the electronic medical record was submitted by an authorized hospital, providing the electronic medical record to the authorized hospital.

7. The method of claim 6, further comprising:
receiving, from the authorized hospital, an updated electronic medical record; and
incorporating the updated electronic medical record into the blockchain.

8. The method of claim 7, further comprising:
determining, by the node, whether the updated electronic medical record contains a prescription; and
in response to a determination that the updated electronic medical record contains a prescription, notifying the patient of the prescription.

9. (canceled)

10. A device for storing and processing electronic medical records, comprising:
one or more processors; and
one or more computer-readable memories coupled to the one or more processors and having instructions stored thereon that are executable by the one or more processors, wherein the one or more processors are configured to:
receive an electronic medical record for a patient;
incorporate the electronic medical record into a blockchain maintained by a blockchain system, wherein the one or more processors are further configured to:
include data corresponding to the electronic medical record in a data block;
broadcast the data block in the blockchain system;
determine whether the data block is accepted in the blockchain system; and
in response to a determination that the data block is accepted in the blockchain system, add the data block to the blockchain;
determine whether the electronic medical record contains a prescription;
in response to a determination that the electronic medical record contains a prescription, notify the patient of the prescription;
receive an updated prescription status;
incorporate the updated prescription status into the blockchain; and
execute a smart contract recorded on the blockchain to update the electronic medical record based on the updated prescription status to indicate whether refill of the prescription is available.

11. (canceled)

12. A non-transitory computer-readable medium having stored therein instructions that, when executed by a processor of a device, cause the device to perform a method for storing and processing electronic medical records, the method comprising:
receiving an electronic medical record for a patient;
incorporating the electronic medical record into a blockchain maintained by a blockchain system, the incorporating comprising;
including data corresponding to the electronic medical record in a data block;
broadcasting the data block in the blockchain system;
determining whether the data block is accepted in the blockchain system; and
in response to a determination that the data block is accepted in the blockchain system, adding the data block to the blockchain;
determining whether the electronic medical record contains a prescription;
in response to a determination that the electronic medical record contains a prescription, notifying the patient of the prescription;
receiving an updated prescription status;
incorporating the updated prescription status into the blockchain; and
executing a smart contract recorded on the blockchain to update the electronic medical record based on the updated prescription status to indicate whether refill of the prescription is available.

13. The device of claim 10, wherein the one or more processors are further configured to:
receive a request to retrieve the prescription;
determine whether the request to retrieve the prescription was submitted by an authorized pharmacy; and
in response to a determination that the request to retrieve the prescription was submitted by an authorized pharmacy, provide the prescription to the authorized pharmacy.

14. (canceled)

15. (canceled)

16. The device of claim 10, wherein the smart contract invalidates the prescription contained in the electronic medical record when the updated prescription status indicates the prescription contained in the electronic medical record has been filled completely and no refill prescription remains.

17. The device of claim 10, wherein the one or more processors are further configured to:
receive a request to retrieve the electronic medical record;
determine whether the request to retrieve the electronic medical record was submitted by an authorized hospital; and
in response to a determination that the request to retrieve the electronic medical record was submitted by an authorized hospital, provide the electronic medical record to the authorized hospital.

18. The device of claim 17, wherein the one or more processors are further configured to:
receive, from the authorized hospital, an updated electronic medical record; and
incorporate the updated electronic medical record into the blockchain.

19. The device of claim 18, wherein the one or more processors are further configured to:
determine whether the updated electronic medical record contains a prescription; and
in response to a determination that the updated electronic medical record contains a prescription, notify the patient of the prescription.

20. (canceled)
